(19) United States
(12) Patent Application Publication
(10) Pub. No.: US 2021/0228365 A1
(43) Pub. Date: Jul. 29, 2021

(54) TECHNIQUE FOR ADDRESSING FOCAL DEFECTS OF ARTICULAR CARTILAGE

(71) Applicant: Zimmer, Inc., Warsaw, IN (US)

(72) Inventor: Andrew Rolfe Hopkins, Winterthur (CH)

(21) Appl. No.: 17/153,177

(22) Filed: Jan. 20, 2021

Related U.S. Application Data
(60) Provisional application No. 62/965,567, filed on Jan. 24, 2020.

(51) Int. Cl. A61F 2/30; A61F 2/40

(52) U.S. Cl. CPC A61F 2/30756; A61F 2/40

(57) ABSTRACT

A system can include a plurality of grouter devices configured to lodge in one or more focal defects located on a pair of articular surfaces; and a plurality of buffer devices configured to fill a space between the pair of articular surfaces; wherein the plurality of grouter devices and the plurality of buffer devices are sized and shaped such that a contact pressure between the pair of articular surfaces is diminished.

### CLAIM OF PRIORITY

This application claims the benefit of priority under 35 U.S.C. § 119(e) of U.S. Provisional Patent Application Ser. No. 62/965,567, filed on Jan. 24, 2020, which is herein incorporated by reference in its entirety

### FIELD

The present subject matter relates generally to a medical system, and more specifically to addressing focal defects in articular cartilage.

### BACKGROUND

Articular damage due to wear and tear, commonly referred to as osteoarthritis (OA), is one of the principal reasons for surgical treatment using orthopaedic devices. The onset of OA can be accompanied by joint pain, increasing in intensity, but initially manageable using pain-killers. Once the use of self-medicated pain-killers has become frequent, or their effectiveness has begun to diminish, the individual faces the prospect of medically assisted pain management using injections, or surgery.

For a younger patient, or those unable to have any surgical intervention, it is not appropriate to consider a joint replacement. Besides of the impact that such a surgery and its rehabilitation would have on the lifestyle and capacity to work for the individual, the potential for further surgeries to be needed increases with a higher demand and longer living patient.

As a result, while there are treatment modalities at the ends of the spectrum of joint disorder, there are limited non-surgical and low-impact options for the intervening period. In the case of some individuals, this interval might last many years, and will have direct or indirect consequences to the individual's social and/or professional life.

### OVERVIEW

Example 1 can include subject matter that can include a system including a plurality of grouter devices configured to lodge in one or more focal defects located on a pair of articular surfaces; and a plurality of buffer devices configured to fill a space between the pair of articular surfaces; wherein the plurality of grouter devices and the plurality of buffer devices are sized and shaped such that a contact pressure between the pair of articular surfaces is diminished.

In Example 2, the subject matter of Example 1 can optionally include the plurality of grouter devices being flat devices defining an oval shape.

In Example 3, the subject matter of Example 2, can include the oval shape of the plurality of grouter devices having a length of between about 200 μm to about 500 μm and a width of about 50 μm to about 100 μm.

In Example 4, the subject matter of Example 3 can include the plurality of grouter devices having a thickness of about 10 μm to about 50 μm.

In Example 5, the subject matter of any of Examples 1-4 can include the plurality of grouter devices being configured to fill a space defined by the focal defect such that the plurality of grouter devices provide load-sharing with the native cartilage on the pair of articular surfaces.

In Example 6, the subject matter of any of Examples 1-5 can include the plurality of buffer devices being spherical.

In Example 7, the subject matter of Example 6 can include the plurality of buffer devices having a diameter of about 200 μm to about 500 μm.

In Example 8, the subject matter of any of Examples 1-7 can include the plurality of buffer devices being configured to define an inter-positional layer between the pair of articular surfaces such that the plurality of buffer devices act as an inter-articular cushion.

In Example 9, the subject matter of any of Examples 1-8 can include the plurality of grouter devices and the plurality of buffer devices being injectable hydrogels.

In Example 10, the subject matter of Example 9 can include the injectable hydrogels include one or more therapeutic agents.

Example 11 can include subject matter that can include a method to relieve pain in a joint space between a pair of articular surfaces, the method including injecting into the joint space a plurality of grouter devices configured to lodge in one or more focal defects located on the pair of articular surfaces; and injecting into the joint space a plurality of buffer devices configured to fill the joint space between the pair of articular surfaces; wherein the plurality of grouter devices and the plurality of buffer devices are sized and shaped such that a contact pressure between the pair of articular surfaces is diminished.

In Example 12, the subject matter of Example 11 can further include the patient moving the joint through a plurality of motions to enable the plurality of grouter deices to move into the focal defects.

In Example 13, the subject matter of any of Examples 11-12 can further include the patient moving the joint through a plurality of motions after injection of the plurality of buffer devices such that the buffer devices migrate to the intended position between the pair of articular surfaces.

In Example 14, the subject matter of any of Examples 11-13 can include the plurality of grouter devices being flat devices defining an oval shape.

In Example 15, the subject matter of Example 14 can include the oval shape of the plurality of grouter devices having a length of between about 200 μm to about 500 μm and a width of about 50 μm to about 100 μm and have a thickness of about 10 μm to about 50 μm.

In Example 16, the subject matter of any of Examples 11-15 can include the plurality of grouter devices being configured to fill a space defined by the focal defect such that the plurality of grouter devices provide load-sharing with the native cartilage on the pair of articular surfaces.

In Example 17, the subject matter of any of Examples 11-16 can include the plurality of buffer devices being spherical and having a diameter of about 200 μm to about 500 μm.

In Example 18, the subject matter of any of Examples 11-17 can include the plurality of buffer devices being configured to define an inter-positional layer between the pair of articular surfaces such that the plurality of buffer devices act as an inter-articular cushion.

In Example 19, the subject matter of any of Examples 11-18 can include the plurality of grouter devices and the plurality of buffer devices being injectable hydrogels.

In Example 20, the subject matter of Example 19 can include the injectable hydrogels including one or more therapeutic agents.

These examples can be combined in any permutation or combination. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a representation of a focal defect in an articular cartilage.

FIG. 2 shows a view of a buffer device, in accordance with one embodiment.

FIG. 3 shows a top view and a side view of a grouter device, in accordance with one embodiment.

FIG. 4 shows a representation of a focal defect after being filled with a plurality of grouter devices, in accordance with one embodiment.

FIG. 5 shows a representation of an injection of a plurality of buffer devices into a joint space, in accordance with one embodiment.

FIG. 6 shows a native joint space before treatment.

FIG. 7 shows the joint space after injection of a plurality of grouter devices into the joint space, in accordance with one embodiment.

FIG. 8 shows the joint space after injection of a plurality of buffer devices into the joint space, in accordance with one embodiment.

FIG. 9 shows a schematic of a method of treating a focal defect, in accordance with one embodiment.

### DETAILED DESCRIPTION

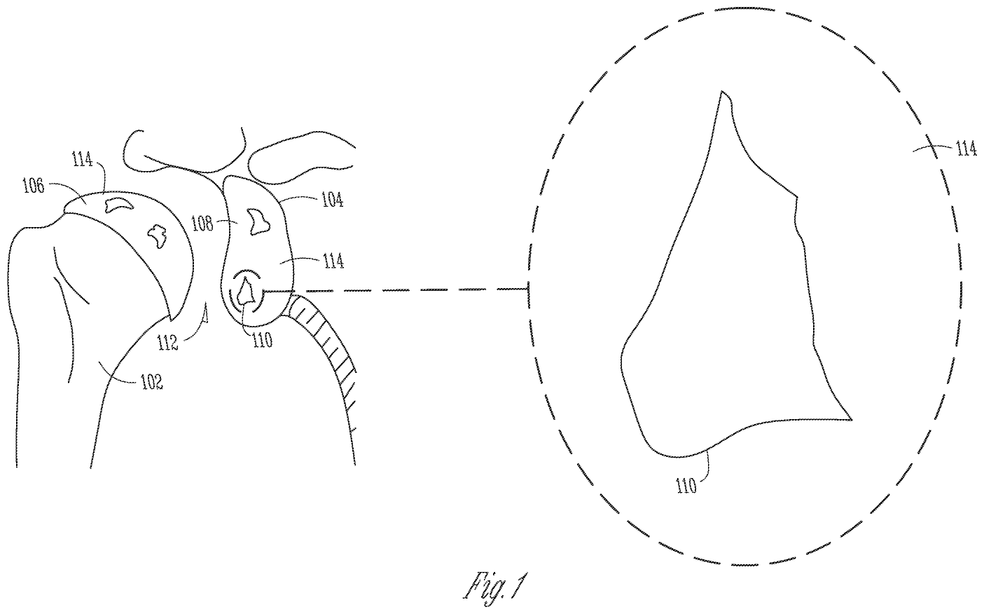

FIG. 1 shows a representation of a focal defect 110 in an articular cartilage 114. In this example the joint shown is a glenohumeral joint. The present system also applies to any other applicable joints in a body, such as the knees, hips, or other articular joints subject to wear of the articular cartilage surfaces.

Accordingly, in this example, the glenohumeral joint includes the humerus 102 having cartilage 114 over a humeral articular surface 106 and a glenoid 104 having cartilage 114 over a glenoid articular surface 108. There is a joint space 112 between the articular surfaces 106, 108 where the surfaces meet and articulate against each other. There can be one or more focal defects 110 in the cartilage 114 of one or both of the articular surfaces 106, 108.

Focal defects, such as focal defects 110, of the articular cartilage 114 may be induced through trauma or wear and tear of the articular cartilage surfaces of the joint over time. These localised defects are associated with pain, primarily due to differential loading or “tugging” forces being applied to the damaged area, essentially resulting in stress concentrations not experienced in normal cartilage.

The current system provides a treatment for the focal defects 110 for patients with early to mid-stage OA, or who are too young for joint replacement, or for patients who cannot allow for a rehabilitation period due to their work/personal situation, but for who pain-killers are beginning to offer diminishing returns.

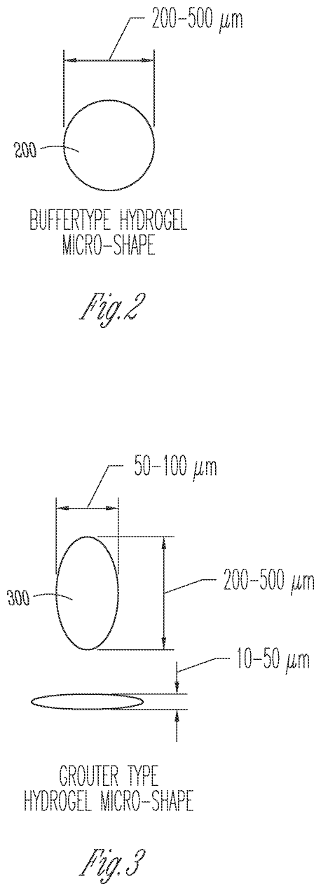

FIG. 2 shows a view of a buffer device 200, in accordance with one embodiment, and FIG. 3 shows a top view and a side view of a grouter device 300, in accordance with one embodiment.

The buffer device 200 and the grouter device 300 can be formed as injectable hydrogel micro-shapes and can be deployed into the affected joint space 112 (FIG. 1) using one or more injections. The two shape types of the buffer device 200 and the grouter device 300 perform different but complementary functions in the joint space.

For example, the grouter device 300 can be flat and relatively oval shaped and a plurality of such grouter devices 300 are intended to make their way, through normal motion of the joint, into the focal defects present in the cartilage. The flat, oval shape of the grouter devices 300 allow the grouter devices 300 to lodge or wedge into the focal defects and fill the space defined by the focal defect, thus off-setting the stress concentrations in the remaining native cartilage by providing load-sharing and reducing the pain experienced during joint motion.

In one example, the oval shape of the plurality of grouter devices 300 can have a length of between about 200 μm to about 500 μm and a width of about 50 μm to about 100 μm. The thickness of the grouter devices 300 can be about 10 μm to about 50 μm.

The buffer device 200 can be spherical and a plurality of such buffer devices 200 are intended to make their way, through normal motion of the joint, to the space in-between the articular surfaces and fulfil the role of an inter-positional layer. The spherical shape of the plurality of buffer devices 200 allow the buffer devices 200 to roll over and between the mixed surfaces of the native cartilage and the grouter devices 300 that occupy the focal defects, offsetting further the stress concentration in the remaining native cartilage by diminishing the contact pressures and acting as an intra-articular cushion. In one example, the plurality of buffer devices 200 can have a diameter of about 200 μm to about 500 μm.

In one example, a material for constructing the grouter devices 300 and the buffer devices 200 can include a hydrogel material having a low elasticity (i.e. compressible and not prone to induce high contact stresses), be biocompatible (long term), can absorb water but with a relatively limited resulting expansion, resistant to early resorption into the body, can be manufactured according to the dimensions discussed above, and can be loaded sterile, in solution, into a syringe which can allow for injection into the joint space.

In one example, the hydrogel material composition of the grouter devices 300 and the buffer devices 200 be adjusted so that during resorption of the devices 200, 300 an anaesthetic, anti-inflammatory, anti-infection or cartilage growth promoting agent, or other therapeutic agent can be released.

One example of a focal defect treatment is now presented, wherein the introduction of the two types of hydrogel micro-shapes are implemented in two separate phases.

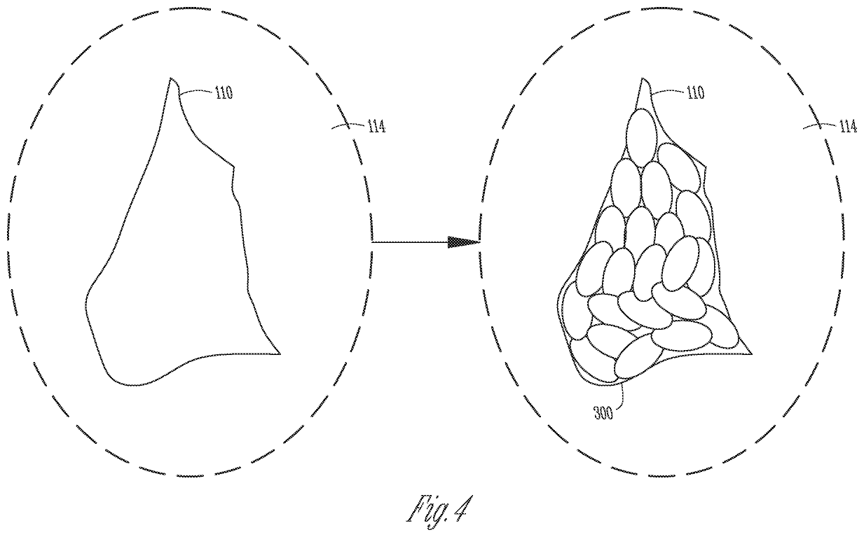

FIG. 4 shows a representation of a focal defect 110 after being filled with a plurality of grouter devices 300, in accordance with one embodiment. Referring also to FIG. 1, a physician can inject a plurality of grouter devices 300 into the joint space 112. After the injection of the grouter devices 300 into the joint space 112, the patient then moves the joint through various arcs of motion in order to enable the grouter devices 300 to move into their respective intended positions in the joint space 112 (i.e. within the focal defects 110 defects of the cartilage 114). As shown in FIG. 4, the flat, oval shape of the grouter devices 300 allows the grouter devices 300 fill and lodge into the edges of the focal defect 110 and also to layer upon each other and overlap. Thus, the flat, oval shape allows the grouter devices 300 to fill a majority of both the area and height of the focal defect 110.

This process might be possible to perform within the timeframe of a clinical appointment but can require several days of normal use of the joint in order to embed the grouter devices 300 into the focal defects 110. Due to the flat cross-section and oval shape of the grouter devices 300, these relatively flexible pieces can embed into the focal defects 110 and essentially grout or fill the gaps, as represented in FIG. 4.

At this stage of the process, there can be an expected reduction in joint pain due to the load offsetting and minimised stress concentrations in the native cartilage due to the grouter devices. The grouter devices 300 remain in-situ due to their geometry (small radius of tips, flat cross section, oval shape) and normal compression forces of the joint.

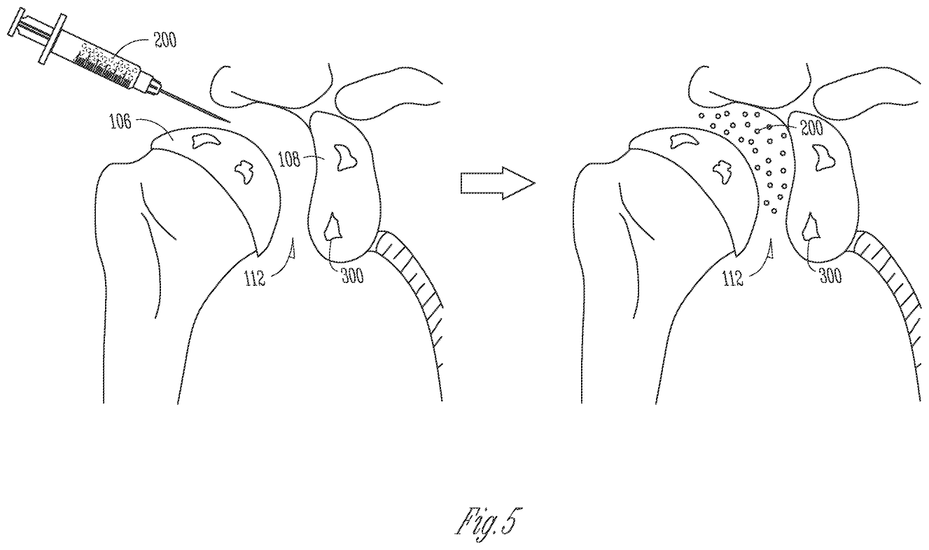

FIG. 5 shows a representation of an injection of a plurality of buffer devices 200 into the joint space 112, in accordance with one embodiment. In one example, following the deployment and positioning of the grouter devices 300, a second phase of the treatment, being the implementation of the buffer devices 200, can proceed.

In this example a second injection is made into the joint space 112 to deliver the buffer devices 200. In general, it is expected that the quantity/volume of buffer devices 200 that should be introduced to the joint space 112 would be larger than the number of grouter devices 300, due to the intended use of these.

Again, after the injection, the patient would need to actively mobilise their joint in order to encourage the buffer devices 200 to migrate to their intended location between the two contacting articulating surfaces 106, 108.

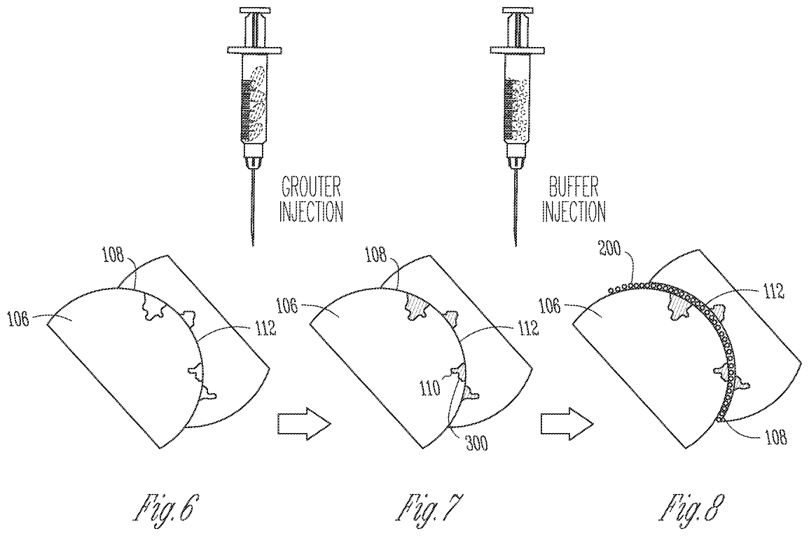

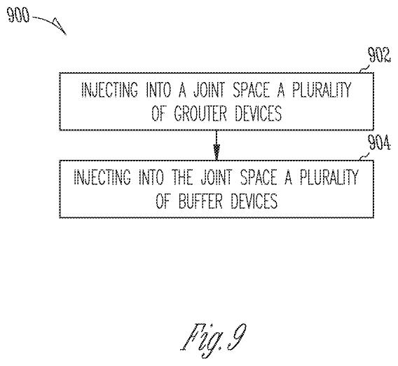

FIGS. 6-8 show a representation of the entire process of focal defect treatment according the present system. FIG. 6 shows a native joint space 112 between the articular surfaces 106, 108 before treatment including the focal defects 110. FIG. 7 shows the joint space 112 after injection of a plurality of grouter devices 300 into the joint space 112 in order to fill the focal defects 110 on the articular surfaces 106, 108, and FIG. 8 shows the joint space 112 after injection of the plurality of buffer devices 200 into the joint space 112, where the buffer devices 200 define an inter-positional layer 202 between the pair of articular surfaces 106, 108 such that the plurality of buffer 200 devices act as an inter-articular cushion.

Thus, in summary the plurality of grouter devices 300 are configured to lodge in one or more focal defects 110 located on the pair of articular surfaces 106, 108, and the plurality of buffer devices 200 configured to fill the joint space 112 between the pair of articular surfaces 106, 108. As described herein, the plurality of grouter devices 300 and the plurality of buffer devices 200 are sized and shaped such that a contact pressure between the pair of articular surfaces 106, 108 is diminished. Specifically, the grouter devices 300 are flat and oval shaped so as to better fill and lodge with the focal defects 110, and the buffer devices 300 are spherical to act as intra-articular cushion within the joint space 112.

FIG. 9 shows a schematic of a method 900 for treating a focal defect, in accordance with one embodiment. The method 900 can include injecting (902) into the joint space 112 a plurality of grouter devices 300 configured to lodge in one or more focal defects 110 located on the pair of articular surfaces 106, 108. The method can further include injecting (904) into the joint space a plurality of buffer devices 200 configured to fill the joint space 112 between the pair of articular surfaces 106, 108. The plurality of grouter devices 300 and the plurality of buffer devices 200 are sized and shaped such that a contact pressure between the pair of articular surfaces 106, 108 is diminished.

As discussed above, the method can include the patient moving the joint through a plurality of motions to enable the plurality of grouter deices 300 to move into the focal defects 110. Also, the patient can move the joint through a plurality of motions after injection of the plurality of buffer devices 200 such that the buffer devices 200 migrate to the intended position between the pair of articular surfaces 106, 108 to define the inter-positional layer 202.

Following treatment, it is expected that the patient will experience a reduction in joint pain that is caused due to focal defects of the cartilage, and this can reduce the likelihood for needing pain-killers to manage daily tasks. The intent of these devices is to offset the symptoms and delay the progression of the OA condition. Over time, it may be necessary to supplement the joint space with additional injections of either more grouter devices 300 or buffer devices 200.

In other example uses of the system described above, treatment can include injection of a plurality of grouter devices 300 only to reduce joint pain, or injection of a plurality of buffer devices 200 only to reduce joint pain, without first injecting the grouter devices 200, or injecting the grouter devices 300 and then within days injection of the buffer devices 200, or a blended injection of both grouter devices 300 and buffer devices 300 and immediate mobilisation of the joint to move these into their intended positions in the joint space

### ADDITIONAL NOTES

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The claimed invention is:

1. A system comprising:
a plurality of grouter devices configured to lodge in one or more focal defects located on a pair of articular surfaces; and
a plurality of buffer devices configured to fill a space between the pair of articular surfaces;
wherein the plurality of grouter devices and the plurality of buffer devices are sized and shaped such that a contact pressure between the pair of articular surfaces is diminished.

2. The system of claim 1, wherein the plurality of grouter devices are flat devices defining an oval shape.

3. The system of claim 2, wherein the oval shape of the plurality of grouter devices has a length of between about 200 μm to about 500 μm and a width of about 50 μm to about 100 μm.

4. The system of claim 3, wherein the plurality of grouter devices have a thickness of about 10 μm to about 50 μm.

5. The system of claim 1, wherein the plurality of grouter devices are configured to fill a space defined by the focal defect such that the plurality of grouter devices provide load-sharing with the native cartilage on the pair of articular surfaces.

6. The system of claim 1, wherein the plurality of buffer devices are spherical.

7. The system of claim 6, wherein the plurality of buffer devices have a diameter of about 200 μm to about 500 μm.

8. The system of claim 1, wherein the plurality of buffer devices are configured to define an inter-positional layer between the pair of articular surfaces such that the plurality of buffer devices act as an inter-articular cushion.

9. The system of claim 1, wherein the plurality of grouter devices and the plurality of buffer devices are injectable hydrogels.

10. The system of claim 9, wherein the injectable hydrogels include one or more therapeutic agents.

11. A method to relieve pain in a joint space between a pair of articular surfaces, the method comprising:
injecting into the joint space a plurality of grouter devices configured to lodge in one or more focal defects located on the pair of articular surfaces; and
injecting into the joint space a plurality of buffer devices configured to fill the joint space between the pair of articular surfaces;
wherein the plurality of grouter devices and the plurality of buffer devices are sized and shaped such that a contact pressure between the pair of articular surfaces is diminished.

12. The method of claim 11, further including the patient moving the joint through a plurality of motions to enable the plurality of grouter devices to move into the focal defects.

13. The method of claim 11, further including the patient moving the joint through a plurality of motions after injection of the plurality of buffer devices such that the buffer devices migrate to the intended position between the pair of articular surfaces.

14. The method of claim 11, wherein the plurality of grouter devices are flat devices defining an oval shape.

15. The method of claim 14, wherein the oval shape of the plurality of grouter devices has a length of between about 200 μm to about 500 μm and a width of about 50 μm to about 100 μm and have a thickness of about 10 μm to about 50 μm.

16. The method of claim 11, wherein the plurality of grouter devices are configured to fill a space defined by the focal defect such that the plurality of grouter devices provide load-sharing with the native cartilage on the pair of articular surfaces.

17. The method of claim 11, wherein the plurality of buffer devices are spherical and have a diameter of about 200 μm to about 500 μm.

18. The method of claim 11, wherein the plurality of buffer devices are configured to define an inter-positional layer between the pair of articular surfaces such that the plurality of buffer devices act as an inter-articular cushion.

19. The method of claim 11, wherein the plurality of grouter devices and the plurality of buffer devices are injectable hydrogels.

20. The method of claim 19, wherein the injectable hydrogels include one or more therapeutic agents.
